(19) United States
(12) Patent Application Publication
(10) Pub. No.: US 2018/0030065 A1
(43) Pub. Date: Feb. 1, 2018

(54) FLUORESCEIN POLYMER CONJUGATES

(71) Applicant: INDICATOR SYSTEMS INTERNATIONAL, INC., Newport Beach, CA (US)

(72) Inventors: Gerald F. Swiss, San Diego, CA (US); Richard Willency, Newport Beach, CA (US); Craig Keshishian, Newport Beach, CA (US); Richard Pariza, Newport, CA (US); Robert M. Moriarty, Michiana Shores, MI (US)

(73) Assignee: ISI Life Sciences, Inc., Newport Beach, CA (US)

(21) Appl. No.: 15/546,639

(22) PCT Filed: Jan. 27, 2016
(86) PCT No.: PCT/US2016/015179

§ 371 Date: Jul. 26, 2017

Related U.S. Application Data
(60) Provisional application No. 62/108,475, filed on Jan. 27, 2015.
(60) Provisional application No. 62/114,495, filed on Feb. 10, 2015.

(51) Int. Cl. C07D 493/10; A61K 49/00; C09B 69/10; C09K 11/06; C08G 77/38; C09B 11/08

(52) U.S. Cl. CPC C07D 493/10; C08G 77/38; C09B 11/08; C09B 69/103; C09K 11/06; A61K 49/0054; A61K 49/0043

(57) ABSTRACT

This technology relates to fluorescein conjugates including fluorescein conjugates of polymers.

### FIELD OF THE INVENTION

This invention relates to polymer conjugates and liposomes that can be used in a variety of products such as catheters where the conjugates and/or liposomes allow for early detection of infection.

### BACKGROUND

Catheters and Infection.

The long-term use of indwelling medical devices, e.g., central venous catheters, is limited by their high risk of infection. Due to the high infection risk, the Infectious Diseases Society of America has developed clinical practice guidelines for the diagnosis and management of intravascular catheter-related infection. [See IDSA Guidelines for Intravascular Catheter-Related Infection, CID, 2009:48.] The IDSA generally recommends that catheter cultures not be obtained routinely, but rather only when removed for suspected catheter-related bloodstream infection. The IDSA's specific recommendations vary depending upon patient characteristic and whether the catheter is short-term or long-term.

Biofilms.

Catheter-related infections are often biofilm-related infections. Biofilms develop when microorganisms irreversibly adhere to the device's surface via extracellular polymers, e.g., polysaccharides. Microbial biofilms facilitate high growth rates of bacteria and are associated with greater resistance to antibiotic treatment due to the diminished rate of transport of antimicrobials to the encased cells. Biofilms may develop on or within the indwelling medical devices, e.g., central venous catheters, peritoneal dialysis catheters, and urinary catheters. [See Biofilms and Device-Associated Infections, Emerging Infectious Diseases, Vol. 7, No. 2, March-April 2001, Pp. 277-281].

Food Wrapping and Spoilage Detection.

Expiration dates are currently placed on food and produce that can spoil. Such dates are inexact predictors of food spoilage. Food spoilage is due to the growth of microbes such as bacteria, yeast or fungi. Prevention and detection of food spoilage is useful in preventing associated illness and death. Chemists, engineers and materials scientists have made advances in food spoilage monitoring. For example, scientists have developed sensing polymer films comprising metal oxide semiconductors that indicate changes of conductivity in the films induced by the adsorption of gases released during food spoilage processes. [See “Electrochemical sensors for food authentication,” Wilson and Wilson's Comprehensive Analytical Chemistry, vol. 49, pp. 755-770, 2007]. Other advanced methods of detecting food spoilage include detecting enzymes with enzyme sensors; however, these methods often use enzyme oxidases which may not be compatible with food safety. [See “Amine oxidase-based flow biosensor for the assessment of fish freshness,” Food Control, vol. 11, pp. 13-18, 2000].

### SUMMARY

The most common bacterial infections associated with catheters are those arising from Staphylococcus aureus (staph) including MRSA. In the case of staph and other bacteria, the formation of a biofilm is associated with a drop in pH (http://mbio.asm.org/content/5/5/e01667-14.full). Using this drop in pH as a metric, pH dependent liposomes loaded with an indicator and embodied in the polymeric matrix of the inner or outer wall of the catheter lumen will undergo designed degradation at a pH less acidic than that required to form the biofilm. Accordingly, the incipient formation of a biofilm on a catheter wall will lead to release of the indicator allowing the clinician to determine the presence of bacteria and the incipient formation of a biofilm.

Accordingly, in one aspect of this invention, a coating is described comprising a pH-dependent liposome loaded with an indicator such as a dye or fluorescent material. The liposome is dispersed within or on a polymeric matrix that is in proton communication with the fluid within the catheter. Such communication includes either the proton itself (Hi) or the precursor molecule such as lactic acid either of which are referred to herein as the “proton”. The liposome comprises lipid wall components that are designed to degrade and release the indicator at a particular narrow pH range. In another aspect, a coating is described, wherein the polymeric matrix further comprises a hydrogel to maximize the communication of protons from the fluid within the catheter to the liposomes within the hydrogel polymer. In another aspect, the pH range for designed degradation is between 5.0 and 6.0. In one aspect, the pH for designed degradation of the liposome is approximately 5.2. In another aspect the pH for designed degradation of the liposome is approximately 6.0.

In another aspect, a catheter for use inside a human patient is described that is coated with a polymeric film on the outer surface, inner surface, or both surfaces of the catheter wherein the polymeric film comprises a pH-dependent liposome loaded with an indicator such as a dye or fluorescent material or where the dye or fluorescent molecule is covalent bound to the polymer of the catheter. As the formation of the biofilm on an interior catheter wall is particularly troublesome as the interior is in direct communication with the patient's blood stream, in a preferred embodiment, the liposome containing polymeric film is loaded onto the interior wall of the catheter so as to be in proton communication with the fluid within the catheter. In another embodiment, the dye or fluorescent molecule is covalently tethered to the polymer wall of the catheter and is selected to provide a detectable signal upon a change in pH. In one embodiment, the dye, fluorescent molecule or pro-fluorescent molecule is covalently tethered to the polymer wall. In such cases, the presence of bacterial growth will result in a color of the dye or fluorescence property of the fluorescent molecule changes. In the case of a pro-fluorescent molecule, the bacterial growth will convert such molecules from non-fluorescent to fluorescent. Such molecules are described herein, such as the fluorescent derivative moieties described below.

In another aspect, the liposome undergoes designed degradation at a particular pH range. In another aspect, the use of biologically compatible food dyes as an indicator will result in a visible color change of the fluid within the catheter upon degradation of the liposome. In another aspect, the polymeric film further comprises a hydrogel. When used in a catheter, a preferred pH for degradation of the liposome is 6.0 to 6.5 as this range is below physiological pH and will readily detect incipient biofilm formation. Biologically compatible food dyes include Blue No. 1 (or brilliant blue), Green No. 3, and Yellow No. 6 (or sunset yellow), typically as sodium salts. In some aspects, the food dyes are used in an amount according to the guidance of a regulatory agency, such as the U.S. Food and Drug Administration (FDA). For example, in some aspects, Blue No. 1 is used in an amount of no more than 12 mg/kg body weight/day, Green No. 3 is used in an amount of no more than 2.5 mg/kg body weight/day, and Yellow No. 6 is used in an amount of no more than 3.75 mg/kg body weight/day.

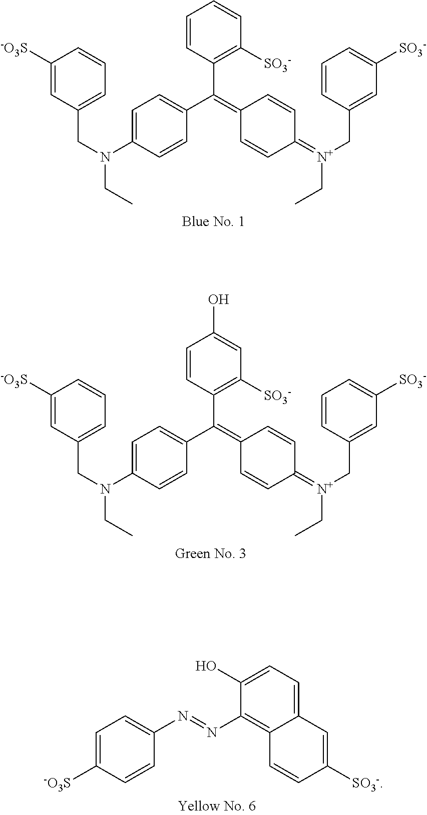

In another aspect, the use of biologically compatible food dyes as an indicator tethered to the polymer of the wall of the catheter will result in a visible color change of the fluid within the catheter at a particular pH range. In another aspect, the polymeric film further comprises a hydrogel. When used in a catheter, a preferred pH for the color change is 6.0 to 6.5 as this range is below physiological pH and will readily detect incipient biofilm formation. Biologically compatible food dyes include Blue No. 1 (or brilliant blue), Green No. 3, and Yellow No. 6 (or sunset yellow), typically as sodium salts. In some aspects, the food dyes are used in an amount according to the guidance of a regulatory agency, such as the U.S. Food and Drug Administration (FDA). For example, in some aspects, Blue No. 1 is used in an amount of no more than 12 mg/kg body weight/day, Green No. 3 is used in an amount of no more than 2.5 mg/kg body weight/day, and Yellow No. 6 is used in an amount of no more than 3.75 mg/kg body weight/day.

In another aspect, a food packaging material is disclosed that comprises a pH-dependent liposome loaded with a food compatible indicator such as a food safe dye dispersed within or on at least a portion of a polymeric matrix wherein the liposome undergoes degradation at a preselected pH range. In one aspect, the food packaging material is disclosed wherein liposomal degradation generates a visible color change to the food. In another aspect, the polymeric film further comprises a hydrogel. In one aspect, the pH range is between 4.8 and 5.2. Alternatively, the food packaging material can contain a dye or fluorescent molecule tethered to the polymer film such that upon such a change in pH, a detectable signal is produced.

In one aspect, provided herein is a compound of Formula V-A or V-A′:

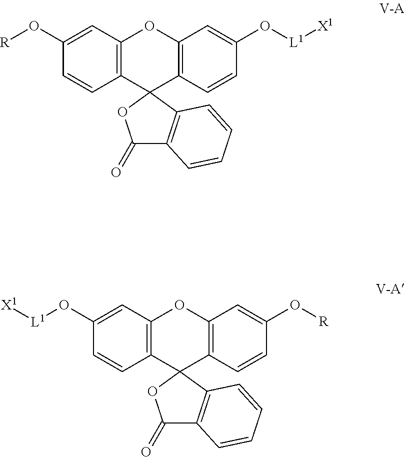

wherein

R is —H, —C(O)(R²⁰), —C(O)O(R²⁰), or —C(O)N(R²¹)₂;

R²⁰ is a C₁-C₃₀ alkyl, C₂-C₃₀ heteroalkyl, C₆-C₁₀ aryl or C₁-C₁₀ heteroaryl;

each R²¹ is independently hydrogen, C₁-C₃₀ alkyl, C₂-C₃₀ heteroalkyl, C₆-C₁₀ aryl or C₁-C₁₀ heteroaryl;

L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein; and

X¹ is a polymer.

In another aspect, provided herein is a compound of Formula V-B:

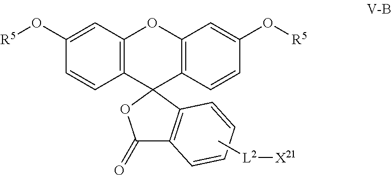

wherein

each R⁵ is independently R or optionally substituted C₁-C₆ alkyl, provided that both R⁵ groups are not optionally substituted C₁-C₆ alkyl;

R is —H, —C(O)(R²⁰), —C(O)O(R²⁰), or —C(O)N(R²¹)₂;

R²⁰ is a C₁-C₃₀ alkyl, C₂-C₃₀ heteroalkyl, C₆-C₁₀ aryl or C₁-C₁₀ heteroaryl;

each R²¹ is independently hydrogen, C₁-C₃₀ alkyl, C₂-C₃₀ heteroalkyl, C₆-C₁₀ aryl or C₁-C₁₀ heteroaryl;

L² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein or L² is a linking group, wherein the linking group is from 1 to 20 carbon atoms and optionally from 1 to 6 heteroatoms selected from the group consisting of oxygen, sulfur, nitrogen, phosphorus and silicon; and

X²¹ is a polymer.

In one aspect, provided herein is a compound of Formula V:

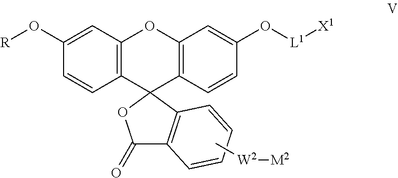

wherein

R is —H, —C(O)(R²⁰), —C(O)O(R²⁰), or —C(O)N(R²¹)₂;

R²⁰ is a C₁-C₃₀ alkyl, C₂-C₃₀ heteroalkyl, C₆-C₁₀ aryl or C₁-C₁₀ heteroaryl;

each R²¹ is independently hydrogen, C₁-C₃₀ alkyl, C₂-C₃₀ heteroalkyl, C₆-C₁₀ aryl or C₁-C₁₀ heteroaryl;

L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein;

X¹ is a polymer;

W² is a linking group, wherein the linking group is from 1 to 20 carbon atoms and optionally from 1 to 6 heteroatoms selected from the group consisting of oxygen, sulfur, nitrogen, phosphorus and silicon or W² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein;

M² is selected from the group consisting of hydrogen, —COOCR¹⁰₃, —COCR¹⁰₃, —C(R¹¹)₃, —C(R¹²)₂—O—R¹³, —COR¹⁴, —Si(R¹⁵)₃, C₁-C₁₀ alkyl group, and a polymer;

each R¹⁰ is independently a C₁-C₆ alkyl optionally substituted with 1, 2 or 3 C₁-C₆ alkoxy groups;

each R¹¹ is independently hydrogen or a phenyl optionally substituted with 1, 2 or 3 substituents independently selected from C₁-C₆ alkyl and C₁-C₆ alkoxy groups;

each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms;

each R¹⁴ is independently a C₁-C₁₀ alkyl; and

each R¹⁵ is independently a C₁-C₃ alkyl.

In another aspect, provided herein is a compound of Formula (I) or (II):

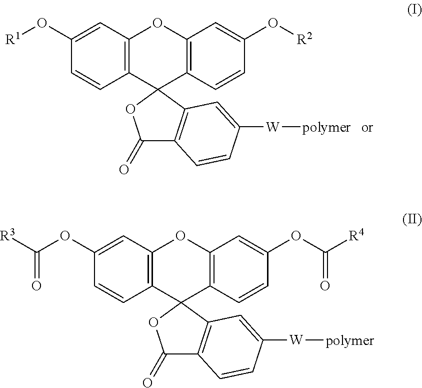

wherein

polymer represents the rest of the polymer molecule,

W is a linking group of from 1 to 20 carbon atoms and optionally from 1 to 6 heteroatoms selected from the group consisting of oxygen, sulfur, nitrogen, phosphorus and silicon,

R¹ and R² are independently H, —COOCR¹⁰₃, —COCR¹⁰₃, —C(R¹¹)₃, —C(R¹²)₂—O—R¹³, —COR¹⁴, or —Si(R¹⁵)₃,

R³ and R⁴ are independently C₁ to C₁₀ alkyl or C₁ to C₁₀ alkoxy,

each R¹⁰ is independently a C₁-C₆ alkyl optionally substituted with 1, 2 or 3 C₁-C₆ alkoxy groups,

each R¹¹ is independently a hydrogen or a phenyl optionally substituted with 1, 2 or 3 substituents independently selected from C₁-C₆ alkyl and C₁-C₆ alkoxy groups, such as methyl and methoxy,

each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms,

each R¹⁴ is independently C₁ to C₁₀ alkyl, and

each R¹⁵ is independently a C₁-C₃ alkyl, such as methyl.

In another aspect, provided herein is a compound of Formula VI-A or VI-A′:

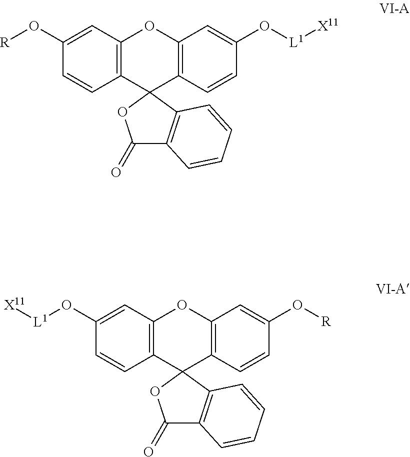

wherein

R is —H, —C(O)(R²⁰), —C(O)O(R²⁰), or —C(O)N(R²¹)₂;

R²⁰ is a C₁-C₃₀ alkyl, C₂-C₃₀ heteroalkyl, C₆-C₁₀ aryl or C₁-C₁₀ heteroaryl;

each R²¹ is independently hydrogen, C₁-C₃₀ alkyl, C₂-C₃₀ heteroalkyl, C₆-C₁₀ aryl or C₁-C₁₀ heteroaryl;

L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein; and

X¹¹ is a reactive functionality that reacts with a complementary group on a polymer.

In another aspect, provided herein is a compound of Formula VI-B

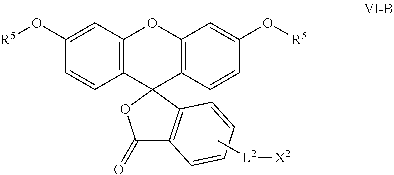

wherein

each R⁵ is independently R or optionally substituted C₁-C₆ alkyl, provided that both R⁵ groups are not optionally substituted C₁-C₆ alkyl;

R is —H, —C(O)(R²⁰), —C(O)O(R²⁰), or —C(O)N(R²¹)₂;

R²⁰ is a C₁-C₃₀ alkyl, C₂-C₃₀ heteroalkyl, C₆-C₁₀ aryl or C₁-C₁₀ heteroaryl;

each R²¹ is independently hydrogen, C₁-C₃₀ alkyl, C₂-C₃₀ heteroalkyl, C₆-C₁₀ aryl or C₁-C₁₀ heteroaryl;

L² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein or L² is a linking group, wherein the linking group is from 1 to 20 carbon atoms and optionally from 1 to 6 heteroatoms selected from the group consisting of oxygen, sulfur, nitrogen, phosphorus and silicon; and

X² is a reactive functionality that reacts with a complementary group on a polymer.

In another aspect, provided herein is a compound of Formula VI:

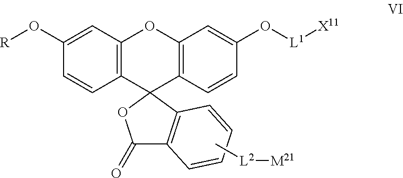

wherein

R²⁰ is a C₁-C₃₀ alkyl, C₂-C₃₀ heteroalkyl, C₆-C₁₀ aryl or C₁-C₁₀ heteroaryl;

each R²¹ is independently hydrogen, C₁-C₃₀ alkyl, C₂-C₃₀ heteroalkyl, C₆-C₁₀ aryl or C₁-C₁₀ heteroaryl;

L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein;

X¹¹ is a reactive functionality that reacts with a complementary group on a polymer;

L² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein or L² is a linking group, wherein the linking group is from 1 to 20 carbon atoms and optionally from 1 to 6 heteroatoms selected from the group consisting of oxygen, sulfur, nitrogen, phosphorus and silicon;

M²¹ is selected from the group consisting of hydrogen, —COOCR¹⁰₃, —COCR¹⁰₃, —C(R¹¹)₃, —C(R¹²)₂—O—R¹³, —COR¹⁴, —Si(R¹⁵)₃, C₁-C₁₀ alkyl group, and a reactive functionality that reacts with a complementary group on a polymer;

each R¹¹ is independently a hydrogen or a phenyl optionally substituted with 1, 2 or 3 substituents independently selected from C₁-C₆ alkyl and C₁-C₆ alkoxy groups;

each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms;

each R¹⁴ is independently a C₁-C₁₀ alkyl; and

each R¹⁵ is independently a C₁-C₃ alkyl.

In another aspect, provided herein is a compound of Formula (III) or (IV):

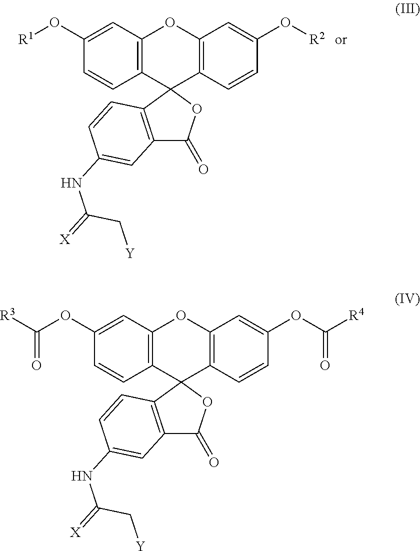

where
X is sulfur or oxygen,
Y is chloro, bromo or iodo,
R¹ and R² are independently H, —COOCR¹⁰₃, —COCR¹⁰₃, —C(R¹¹)₃, —C(R¹²)₂—O—R¹³, —COR¹⁴, or —Si(R¹⁵)₃,
R³ and R⁴ are independently C₁ to C₁₀ alkyl or C₁ to C₁₀ alkoxy,
each R¹⁰ is independently a C₁-C₆ alkyl optionally substituted with 1, 2 or 3 C₁-C₆ alkoxy groups,
each R¹¹ is independently a hydrogen or a phenyl optionally substituted with 1, 2 or 3 substituents independently selected from C₁-C₆ alkyl and C₁-C₆ alkoxy groups, such as methyl and methoxy,
each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms,
each R¹⁴ is independently C₁ to C₁₀ alkyl, and
each R¹⁵ is independently a C₁-C₃ alkyl, such as methyl.

### BRIEF DESCRIPTION OF THE FIGURES

FIG. 1 is a schematic representation of the elements to measure fluorescence on a fluorescein coated catheter.

FIG. 2 shows one embodiment of the arrangement, used to measure the bacteria infested neck of the catheter at the patients entry point.

FIG. 3 shows another embodiment, used to measure the bacteria infested catheter internal within the patient.

### DETAILED DESCRIPTION

### Definitions

As used herein, and in the appended claims, the singular forms “a,” “an” and “the” include plural references unless the context clearly dictates otherwise.

“Administering” or “Administration of” a drug to a patient (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

“Comprising” shall mean that the methods and compositions include the recited elements, but not exclude others. “Consisting essentially of” when used to define methods and compositions, shall mean excluding other elements of any essential significance to the combination for the stated purpose. Thus, e.g., a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transitional terms and phrases are within the scope of this invention.

An “alkyl” as used herein refers to straight chain and branched chain saturated or partially unsaturated alkyl groups having from 1 to 30 carbon atoms, and typically from 1 to 20 carbons or, in some embodiments, from 1 to 18, 1 to 15, 1 to 12, 1 to 10, 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. An example of a partially unsaturated alkyl group is an oleyl group. Other examples of alkyl include C₄-C₃₀, C₆-C₃₀, C₈-C₃₀ and C₁₀-C₃₀ alkyl groups.

A “heteroalkyl” refers to a C₂-C₃₀ alkyl group having wherein 1-5 carbon atoms, are replaced with a heteroatom, preferably, with one or more of —NR³⁰—, —S—, —S(O)—, —S(O₂)—, and —O—, where R³⁰ is hydrogen, C₁-C₆ alkyl, or —C(O)R³¹— where R³¹ is hydrogen, or C₁-C₆ alkyl.

An “alkylene” refers to divalent saturated aliphatic hydrocarbyl groups having from 1 to 25 carbon atoms and, in some embodiments, from 1 to 15 carbon atoms. The alkylene groups include branched and straight chain hydrocarbyl groups, such as methylene, ethylene, propylene, 2-methypropylene, pentylene, and the like.

A“heteroalkylene” refers to alkylene wherein 1-5 carbon atoms, are replaced with a heteroatom, preferably, with one or more of —NR³⁰—, —S—, —S(O)—, —S(O₂)—, and —O—, where R³⁰ is hydrogen, C₁-C₆ alkyl, or —C(O)R³¹— where R³¹ is hydrogen, or C₁-C₆ alkyl.

An “alkoxy” refers to the group —O-alkyl, and includes, by way of example, methoxy, ethoxy, n-propoxy, isopropoxy, n-butoxy, t-butoxy, sec-butoxy, and n-pentoxy.

“Aryl” refers to aromatic aryl groups of from 6 to 10 carbon atoms optionally substituted with from 1 to 3 substituents selected from amino, hydroxyl, nitro, fluoro, chloro, bromo, carboxy, carboxyl ester, and nitro. Such groups include substituted and unsubstituted phenyl and naphthyl.

A carboxylate ester is an ester formed between a —C(O)OH group and an alcohol.

A carbamate is formed between a —N═C═O group and an alcohol.

A heterocyclic ring or heterocyclyl or heterocycle is an aromatic or non-aromatic, mono-, bi-, or tricyclic ring containing 2-12 ring carbon atoms and 1-8 ring heteroatoms selected preferably from N, O, S, and P and oxidized forms of N, S, and P. Aromatic heterocylic rings are sometimes referred to herein as heteroaryl. Non-aromatic heterocycles contain no more than 1 to 3 double bonds. Preferably, the heterocycle contains no more than 3 heteroatoms. A heterocyclic ring includes saturated ring systems and ring systems containing 1-3 double bonds, provided that the ring is non-aromatic. Examples of a heterocyclic ring include but are not limited to an azalactone, oxazoline, piperidinyl, piperazinyl, pyrrolidinyl, tetrahydrofuranyl, and tetrahydropyranyl.

A “keto group” is —C(O)—.

Optionally substituted refers to a group, such as, e.g., an alkyl group, that is unsubstituted or where one or more hydrogen atoms in the group are substituted with a functional group, preferably other than itself. Non-limiting substituting functional groups include hydroxy, amino, carboxyl, —O-alkyl, fluoro, chloro, bromo, iodo, aryl such as phenyl, or substituted aryl, and such other groups, e.g, as disclosed here.

A “reactive functionality that reacts with a complementary group on a polymer” refers to any reactive group known to react with a corresponding group on the polymer to form a covalent bond. Examples include chloro, bromo or iodo reacting with an amino group (NH) or a phenoxide; alcohols or amines reacting with an epoxide; phenoxides reacting with an epoxide; an isocyanate reacting with an amine or alcohol; an isothiocyanate reacting with an amine or alcohol; an alcohol reacting with a carboxylic acid or carboxylic acid chloride/bromide; and the like. As to alcohols, these refer to an —OH group attached to a carbon atom of an alkyl, heteroalkyl, alkylene or heteroalkylene group.

A “polymer” is a long chain of repeating units such as ethylene, propylene, oxyethylene, oxypropylene, silicon, urea, and the like. Such polymers preferably have a number average molecular weight of from 1000 to 2,000,000. So polymers such include polyethylene, polypropylene, polyoxyethylene, polyoxypropylene, polyurea, polysilicon. Such polymers have or can be modified to have reactive functionalities which react with its complementary reactive group. Such reactive functionalities include amino, hydroxyl, carboxyl, and the like. Examples include poly2-hydroxyethylacrylate, polyacrylates, Jeffamines, polyvinyl alcohol, and the likes well known to the skilled artisan.

In one embodiment, a coating or film is described comprising a polymer matrix coating that further comprises liposomes on or in the coating. In one embodiment, the liposome undergoes designed degradation within a specific pH range. A range of liposomes are known in the art and the type of liposome used depends upon the desired pH range for apoptosis of said liposome. The pH range is selected based upon the particular type of bacteria that may form in or on the catheter. In one embodiment, the pH range that induces designed degradation of the liposome within the polymer matrix is preselected by the chemist using lipid forming materials well known in the art. In some embodiments, the polymer-liposome coating is biocompatible. Examples of pH dependent liposomes include those described herein, such as in Example 1.

In some embodiments, the liposome comprises a dye that is released upon liposome degradation. In some embodiments, the dye released is visible to the naked eye. In some embodiments, the dye or indicator is detectible by spectroscopy such as a fluorescent material or a blue dye. In some embodiments, the liposome is integrated into the polymer and, in another embodiment, the liposome is included as a component placed on at least a portion of the surface of the polymer such as by use of an adherent biocompatible mass that includes liposomes.

In one embodiment, a catheter is disclosed comprising the coating described herein. In some embodiments, the coating is on the outer surface, inner surface, or both surfaces of the catheter.

In one embodiment, the liposome undergoes degradation within a specific temperature range. The type of liposome used depends upon the desired temperature range for apoptosis of said liposome.

In one embodiment, the polymer stabilizes the liposome to prevent premature or delayed degradation. In some embodiments, the polymer matrix comprises multiple types of polymers. In some embodiments, the polymer matrix comprises only one polymer, e.g., polyacrylamide. In some embodiments, the polymers are optionally natural or synthetic polymers such polyethylene glycol (PEG), chitosan, silk-fibroin, and polyvinyl alcohol (PVA). The polymers are optionally hydrophilic or hydrophobic. In some embodiments, natural and/or synthetic biodegradable polymeric systems are used, such as chitosan, collagen, gelatin, fibrin, alginate, dextran, carbopol, and polyvinyl alcohol. In some embodiments, a naturally occurring linear polysaccharide is used for the polymer matrix, e.g., alginate. The chemical structure of alginate is composed of (1-4)-b-D-mannuronic acid (M) and (1-4)-a-L-guluronic acid (G) units in the form of homopolymeric (MM- or GG-blocks) and heteropolymeric sequences (MG or GM-blocks). In one embodiment, the polymer scaffold comprises dextran; there are two commercial preparations available, namely dextran 40 kilodaltons (kDa) (Rheomacrodex) and dextran 70 Kilodaltons (kDa) (Macrodex).

In some embodiments, the polymer matrix further comprises a hydrogel. In one embodiment, the hydrogel is formed from any protein-based biomaterial, e.g., gelatin. In some embodiments, a hydrogel used in the polymer scaffold is chitosan-based. In some embodiments, the polymer scaffold is biocompatible and does not induce any adverse response when placed in contact with a biological system. In some embodiments, a hydrogel Carbopol formulation is used, which is a synthetic type of hydrogel, e.g., Carbopol 980, Carbopol 974NF resin, and Carbopol 940. In some embodiments, the polymer scaffold comprises polyvinyl alcohol (PVA), which is a water soluble highly hydrophilic synthetic polymer, with a molecular mass of 80 killodaltons (KDa).

In another aspect, the polymer or the hydrogel comprises one or more fluorescent derivative moiety that is non-fluorescent at a pH of higher than 5.5 and/or in the absence of an esterase, but becomes fluorescent when hydrolyzed at a pH of lower than 5.4 or by an esterase such as microbe esterase. Preferably, the polymer contains a pro-fluorescent moiety such as an ester that will become fluorescent in the presence of esterases, lipases and other enzymes expressed by a microorganism. Pro-fluorescent moieties that are capable of being bound (preferably covalently bound) to polymers (including hydrogels and non-hydrogels) are within the scope of this invention.

In some embodiments, the pro-fluorescent bound polymers are of Formula V-A or V-A′:

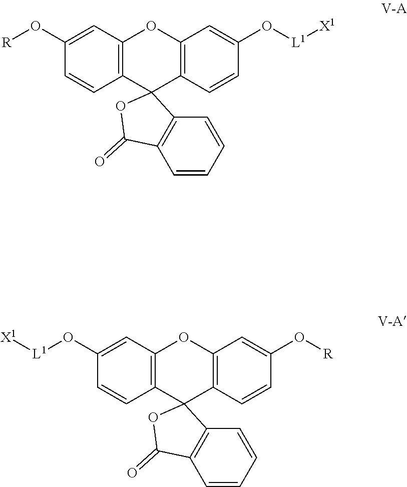

wherein

R is —H, —C(O)(R²⁰), —C(O)O(R²⁰), or —C(O)N(R²¹)₂;

R²⁰ is a C₁-C₃₀ alkyl, C₂-C₃₀ heteroalkyl, C₆-C₁₀ aryl or C₁-C₁₀ heteroaryl;

each R²¹ is independently hydrogen, C₁-C₃₀ alkyl, C₂-C₃₀ heteroalkyl, C₆-C₁₀ aryl or C₁-C₁₀ heteroaryl;

L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein; and

X¹ is a polymer.

In some embodiments, the pro-fluorescent polymer is of Formula V-B:

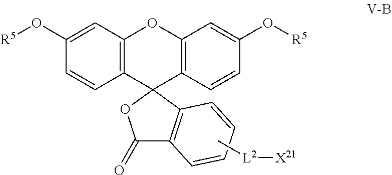

wherein

each R⁵ is independently R or optionally substituted C₁-C₆ alkyl, provided that both R⁵ groups are not optionally substituted C₁-C₆ alkyl;

R is defined as above;

L² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein or L² is a linking group, wherein the linking group is from 1 to 20 carbon atoms and optionally from 1 to 6 heteroatoms selected from the group consisting of oxygen, sulfur, nitrogen, phosphorus and silicon; and

X²¹ is a polymer.

In some embodiments, the pro-fluorescent polymer is of Formula V-B′ or V-B″:

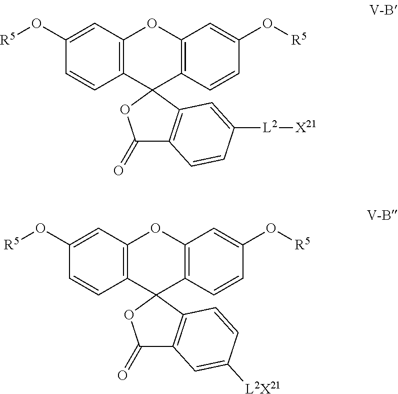

wherein the variables are defined as above.

In some embodiments, the fluorescent derivative moiety is of Formula V:

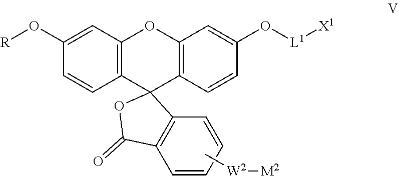

wherein

R, L¹ and X¹ are as defined above;

W² is a linking group, wherein the linking group is from 1 to 20 carbon atoms and optionally from 1 to 6 heteroatoms selected from the group consisting of oxygen, sulfur, nitrogen, phosphorus and silicon or W² is C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate as defined herein;

M² is selected from the group consisting of hydrogen, —COOCR¹⁰₃, —COCR¹⁰₃, —C(R¹¹)₃, —C(R¹²)₂—O—R¹³, —COR¹⁴, —Si(R¹⁵)₃, C₁-C₁₀ alkyl group, and a polymer;

each R¹⁰ is independently a C₁-C₆ alkyl optionally substituted with 1, 2 or 3 C₁-C₆ alkoxy groups;

each R¹¹ is independently hydrogen or a phenyl optionally substituted with 1, 2 or 3 substituents independently selected from C₁-C₆ alkyl and C₁-C₆ alkoxy groups;

each R¹² is independently hydrogen or a C₁-C₆ alkyl and each R¹³ is independently hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms;

each R¹⁴ is independently a C₁-C₁₀ alkyl; and

each R¹⁵ is independently a C₁-C₃ alkyl.

In some embodiments, W² is a linker and M² is a polymer. In some preferred embodiments, M² is a non-polymer substituent.

In some embodiments, the fluorescent derivative moiety is of the Formula (I) or (II):

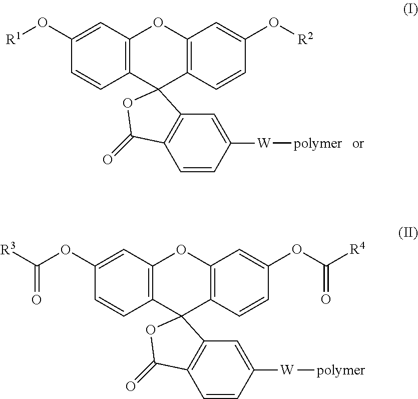

wherein

polymer represents the rest of the polymer molecule,

W is a linking group of from 1 to 20 carbon atoms and optionally from 1 to 6 heteroatoms selected from the group consisting of oxygen, sulfur, nitrogen, phosphorus and silicon,

R¹ and R² are independently H, —COOCR¹⁰₃, —COCR¹⁰₃, —C(R¹¹)₃, —C(R¹²)₂—O—R¹³, —COR¹⁴, or —Si(R¹⁵)₃,

R³ and R⁴ are independently C₁ to C₁₀ alkyl or C₁ to C₁₀ alkoxy,

each R¹⁰ is independently a C₁-C₆ alkyl optionally substituted with 1, 2 or 3 C₁-C₆ alkoxy groups,

each R¹¹ is independently a hydrogen or a phenyl optionally substituted with 1, 2 or 3 substituents independently selected from C₁-C₆ alkyl and C₁-C₆ alkoxy groups, such as methyl and methoxy,

each R¹² is independently hydrogen or a C₁-C₆ alkyl and each R¹³ is independently a hydrogen, C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms,

each R¹⁴ is independently C₁ to C₁₀ alkyl, and

each R¹⁵ is independently a C₁-C₃ alkyl, such as methyl.

In another aspect, the polymer or the hydrogel comprising a pro-fluorescent derivative moiety described above is prepared by reacting a compound of Formula VI-A or VI-A with a polymer to form the compound of Formula V-A or Formula V-B respectively.

Provided herein are compounds of Formula VI-A or VI-A′:

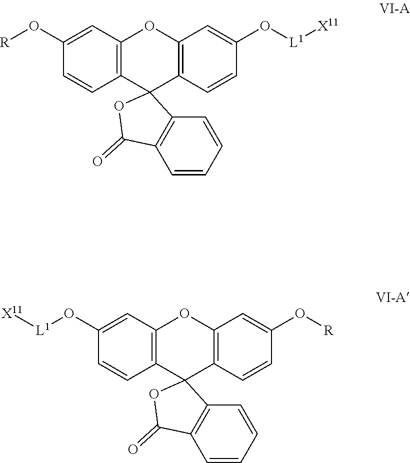

wherein

R and L¹ are as defined above; and

X¹¹ is a reactive functionality that reacts with a complementary group on a polymer.

Provided herein are compounds of Formula VI-B

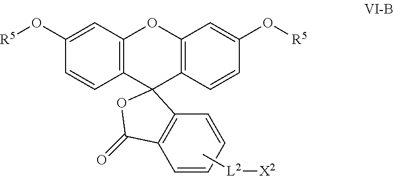

wherein

each R⁵ is as defined above;

L² is a C₁-C₃₀ alkylene or C₂-C₃₀ heteroalkylene optionally substituted with 1, 2 or 3 hydroxy groups, wherein the ydroxyl groups can be further derivatized to an alkoxy, a carboxylate ester, a keto, or a carbamate as defined herein or L² is a linking group, wherein the linking group is from 1 to 20 carbon atoms and optionally from 1 to 6 heteroatoms selected from the group consisting of oxygen, sulfur, nitrogen, phosphorus and silicon;

X² is a reactive functionality that reacts with a complementary group on a polymer.

In another aspect, the polymer or the hydrogel comprising a pro-fluorescent derivative moiety described above is prepared by reacting a compound of Formula VI with a polymer to form the polymer of Formula V. Accordingly, in one embodiment, provided herein are compounds of Formula VI:

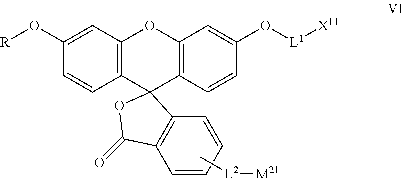

wherein

R, L¹, and L² are defined as above;

X¹¹ is a reactive functionality that reacts with a complementary group on a polymer;

M²¹ is selected from the group consisting of hydrogen, —COOCR¹⁰₃, —COCR¹⁰₃, —C(R¹¹)₃, —C(R¹²)₂—O—R¹³, —COR¹⁴, —Si(R¹⁵)₃, C₁-C₁₀ alkyl group, and a reactive functionality that reacts with a complementary group on a polymer;

each R¹¹ is independently a hydrogen or a phenyl optionally substituted with 1, 2 or 3 substituents independently selected from C₁-C₆ alkyl and C₁-C₆ alkoxy groups;

each R¹² is independently hydrogen or a C₁-C₆ alkyl and each R¹³ is independently a hydrogen or a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms;

each R¹⁴ is independently a C₁-C₁₀ alkyl; and

each R¹⁵ is independently a C₁-C₃ alkyl.

In some embodiments, M²¹ is a reactive functionality that reacts with a complementary group on a polymer. In some preferred embodiments, M²¹ is a group other than a reactive functionality that reacts with a complementary group on a polymer.

In some embodiments, R is H. In some embodiments, R is —C(O)(R²⁰). In some embodiments, R is —C(O)O(R²⁰). In some embodiments, R is —C(O)N(R²¹)₂.

In a preferred embodiment, R²⁰ is a C₄-C₃₀ alkyl. In a more preferred embodiment, R²⁰ is a C₈-C₃₀ alkyl. In an even more preferred embodiment, R²⁰ is a C₁₀-C₃₀ alkyl, most preferably C₁₂-C₃₀ alkyl.

In a preferred embodiment, R²¹ is a C₄-C₃₀ alkyl. In a more preferred embodiment, R²¹ is a C₈-C₃₀ alkyl. In an even more preferred embodiment, R²¹ is a C₁₀-C₃₀ alkyl, most preferably C₁₂-C₃₀ alkyl.

In another aspect, the polymer or the hydrogel comprising a pro-fluorescent derivative moiety described above is prepared by reacting a compound of Formula (III) or (IV) with a polymer to form the polymer of Formula (I) or (II), respectively. The compound of Formula (III) or (IV) are:

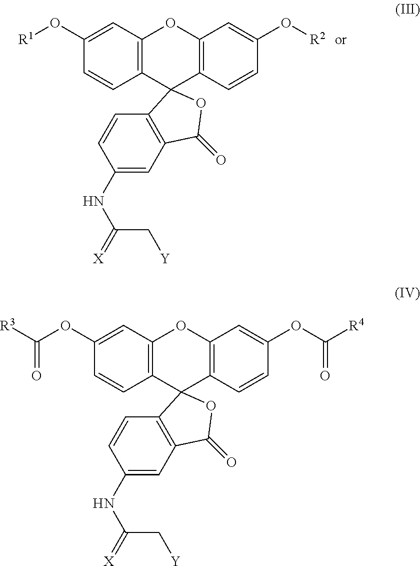

where
X is sulfur or oxygen,
Y is chloro, bromo or iodo,
R¹ and R² are independently H, —COOCR¹⁰₃, —COCR¹⁰₃, —C(R¹¹)₃, —C(R¹²)₂—O—R¹³, —COR¹⁴, or —Si(R¹⁵)₃,
R³ and R⁴ are independently C₁ to C₁₀ alkyl or C₁ to C₁₀ alkoxy,
each R¹⁰ is independently a C₁-C₆ alkyl optionally substituted with 1, 2 or 3 C₁-C₆ alkoxy groups,
each R¹¹ is independently a hydrogen or a phenyl optionally substituted with 1, 2 or 3 substituents independently selected from C₁-C₆ alkyl and C₁-C₆ alkoxy groups, such as methyl and methoxy,
each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms,
each R¹⁴ is independently C₁ to C₁₀ alkyl, and
each R¹⁵ is independently a C₁-C₃ alkyl, such as methyl.

In some aspects, R¹ and R² are independently —C(R¹¹)₃, and each R¹¹ is independently a phenyl optionally substituted with one methyl group. In some aspects, R¹ and R² are independently selected from —CO—O—C(CH₃)₃, —OCH₂—O—CH₃, (p-methoxyphenyl)-diphenylmethyl ether 4′-methoxytrityl (MMTr), di-(p-methoxyphenyl)phenylmethyl ether (4′,4′-dimethoxytrityl or DMTr), tri-(p-methoxyphenyl)methyl ether (4′,4′,4′-trimethoxytrityl

### or TMTr) and

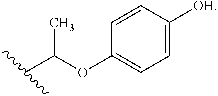 [SMILES: *C(C)Oc1ccc(O)cc1]

In some aspects, R³ and R⁴ are independently methyl, methoxy, t-butyl or t-butoxy.

In some embodiments, R¹ and R² are non-hydrogen substituents.

The compounds provided herein can be prepared from commercially available starting material, such as fluorescein, following well known transformations, such as those illustrated without limitation herein.

### Liposome Size

In some embodiments, liposomes with a size range of 500-5000 nm are used. In other embodiments, nanosized liposomes or small unilamellar vesicles with a size range of 20-50 nm are used. The size of the desired liposome is optionally tuned for the particular type of catheter used and the amount of dye or fluorescent material that needs to be encapsulated.

### Preparation of the Polymer-Liposome Matrix

In some embodiments, the liposome-polymer matrix is formed by inclusion of the liposome within a polymer such as a hydrogel as provided by pH dependent liposomes are well known and can be entrapped into a hydrogel as conducted by conventional means. See, for example, (https://www.google.com/#q=can+liposomes+be+entrapped+in+a+hydrogel). Also see http://kmim.wm.pwr.edu.pl/wp-content/uploads/2014/06/IWBBIO-Bajgrowicz.pdf. Both of which are incorporated herein by reference in their entirety.

In other embodiments, polymer or polymer/hydrogel scaffolds are prepared before inclusion of the liposome and the liposome in placed on the surface of at least a portion of the polymer or polymer/hydrogel. Such placement can be by solvent casting techniques provided that the solvent does not degrade the polymeric scaffold or by stamping the appropriate surface of the scaffold with an adherent mass containing liposomes (e.g., microdots). In some embodiments, the liposome is incorporated into the polymer or polymer-hydrogel scaffold in situ. In some embodiments, the polymer or polymer-hydrogel scaffold is not isolated before the liposome is incorporated. In some embodiments, fabrication of polymer-hydrogel scaffolds suitable for liposome integration are optionally prepared using known techniques, including, but not limited to, fiber bonding, emulsion freeze drying, solvent casting, high-pressure processing, gas foaming, and electrospinning.

In some embodiments, the hydrogel is prepared with a cross-linking agent such as glutaraldehyde or with different types of divalent and polyvalent anions. In some embodiments, fabrication of a chitosan scaffold matrix is achieved using a lyophilization technique. In one embodiment, a chitosan scaffold matrix is formed from crosslinked agents of chitosan solution/hydrogels followed by incubation in liquid nitrogen, or by employing liquid carbon dioxide, solid-liquid separation, or, supercritical immersion precipitation techniques.

In some embodiments, fabrication of alginate hydrogels is achieved via a reaction with cross-linking agents such as divalent or trivalent cations, calcium ions, water-soluble carbodiimide, and/or glutaraldehyde.

In some embodiments, carboxymethyldextran is the hydrogen and is derived by aldehyde-modification or carboxymethylcellulose. In some embodiments, the polymeric-based materials are fabricated using a two phase system, the first phase is water and poly(ethylene glycol) and the second phase is water methacrylated dextran.

In some embodiments, PVA-based hydrogel or scaffolds are fabricated using chemical cross-linking agents such as a citric acid derivative, glutaraldehyde, and formaldehyde, or by physical cross-linking processes such as ultraviolet photo-cross-linking, freezing-thawing, and radiation. Such cross-linking facilitates bonding of a hydrophilic polymer with a hydrophobic polymer thereby enhancing their integrity during use (i.e., the polymer films will not split apart). See, for example, Membranes, 2012, 2, 40-69, which is incorporated herein by reference in its entirety.

In some embodiments, the liposome is first loaded with the indicator such as a dye or other spectroscopically reactive molecule prior to loading it into the polymeric matrix. For example, the liposome is loaded with dye prior to mixing into a fibrinogen solution. The resulting mixture is then injected into porous chitosan films. In another embodiment, the loaded liposomes are incorporated within a polymeric-based system with agitation and subsequent lyophilisation. In some embodiments, loading of liposomes by transmembrane gradients is used.

### Release of the Liposome

Liposome preferably are not released from polymeric-based systems before degradation and such can be predicated upon a variety of factors, including, but not limited to, mesh size of the matrix, size of liposome, steric factors, diffusion, pH, and/or enzyme factor. Detection of a degraded liposome can be made spectroscopically or visibly. Spectroscopic detection includes the detection of the color of food dye used or detection of the fluorescent emission from a fluorescent dye. Such techniques are well known in the art. In one embodiment, a spectrometer is attached to the catheter to consistently monitor and/or record the color or fluorescent emission and/or changes thereof.

### Use in Catheters or Food Spoilage Applications

The liposome/polymer systems described herein have numerous applications. In some embodiments, the liposome/polymer film is coated on the inner or outer surface of a catheter. In some embodiments, the liposome/polymer film is incorporated into traditional food packaging materials.

In embodiments applied to catheters, numerous types of catheters are known and suitable for this application, including, but not limited to central venous catheters, peritoneal dialysis catheters, and urinary catheters. In some embodiments, the catheter further comprises a pH dependent liposome entrapped in a polymer layer such as a hydrophilic polymer. The liposome contains an indicator element such as an entrapped biologically compatible dye, a fluorescent entity or other suitable indicator such a pH indicator. In the presence of an active microbial growth, the indicator will produce a detectable signal. In one embodiment, the signal is a release of the dye from the pH-sensitive liposome as the active microbial growth produces acid components due to aerobic growth (carbon dioxide which combines with water to form carbonic acid). In the case of fluorescence, the fluorescent molecules entrapped in the liposome can be co-entrapped with a fluorescence quencher such that the quencher inhibits fluorescence when the two are in intimate proximity. Rupture of the liposome due to the pH change described above will result in the release of both the fluorescent molecule and the quencher from the liposome thereby inhibiting the action of the quencher and allowing the fluorescent molecule to fluoresce. This provides for the detectible signal that alerts a patient or physician that a bacterial infection is growing in the catheter.

In some embodiments, the liposome/polymer system is incorporated into the traditional packaging of perishable food. In some embodiments, the packaging changes color when the liposomes undergo pH-dependent degradation using the indicators described above. This alerts a consumer or food producer that the food is spoiled. The use of such liposome/polymer systems is particular useful in any environment that produces acid components such as an anaerobic environment that produces lactic acid.

In yet another embodiment, the indicator or fluorescent molecule can be tethered to the polymer film either by direct bonding or through a linking group. Examples of such tethering include the use of a reactive group on the polymer film such as a hydroxyl, carboxyl or amino group which can bind to its complementary functionality on the indicator or fluorescent molecule. Such complementary functional groups are well known and include, by way of example only, alkylhalo groups which react with amines, isocyanates which can react with amines and hydroxyl groups, isothiocyanates which can react with amines an hydroxyl groups, carboxylic acids which can react with amines, hydroxyl or carboxyl acid groups. Complementary functional groups refer to those groups which will react with each other to form a covalent bond. One or both of the complementary functional groups can be attached to a linker moiety that links the functional group to the polymer or to the indicator/fluorescent molecule. Preferred linkers have from 1 to 20 carbon atoms and optionally from 1 to 6 heteroatoms selected from the group consisting of oxygen, sulfur, nitrogen, phosphorus and silicon. The heteroatoms can be included in any art recognized structural motif such as a hydroxyl group, an amine group, a carboxylic acid group, a ketone, an aldehyde, a silicon ester, a phosphate, and the like.

Suitable polymers include those have complementary functional groups attached thereto such as poly(2-hydroxyethyl)methacrylate (poly HEMA), polyvinyl alcohol, copolymers of vinyl alcohol and ethylene, etc.

In one embodiment, the fluorescent molecule employed is any fluorescent molecule that emits a detectable signal upon a change in pH. Such a detectable signal can include a change in emission of the fluorescent molecule upon a change in pH; a change in structure of the compound such that the structural change allows the molecule to fluoresce whereas the molecule before the structural change is non-fluorescent, and the like.

### Optical Detection of Flourescein Coated Catheter

In one embodiment, an L.E.D. element can be mounted on a bandage surface facing the skin and adjacent to a catheter that is entering the patient to excite the flourescein to fluoresce and a multi-mega pixel electronic camera can be mounted on the bandage adjacent to the catheter to measure the fluorescence emitted by the catheter.

In one embodiment, both the L.E.D. and the camera will be controlled by a microprocessor, which will turn on the L.E.D. and which will analyze the camera signal to determine if the signal from the camera is properly detecting fluorescence from the catheter.

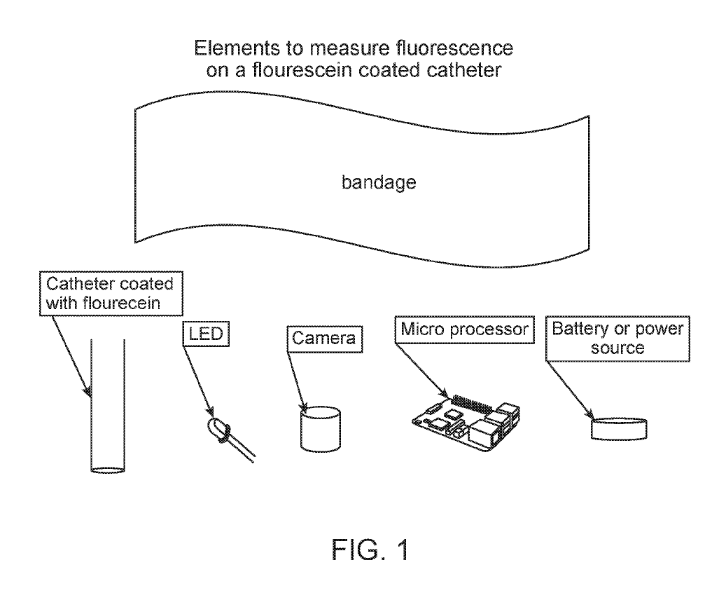

The items/devices needed to detect if the flourescein coated catheter is de-esterified are schematically illustrated in FIG. 1, which is further explained below.

L.E.D.: Light Emitting Diode emits 493 nanometers (blue) light or any wavelength that will cause the flourescein to flouresce when electrically energized.
Digital Camera: containing sufficient pixels to be able to sense the shape of the catheter and the RGB color generated by the flourescein.
Microprocessor: Used to energize the L.E.D., and camera, containing a blue tooth generator, a memory bank to store shape and color information of the catheter and the flourescein.
Power Source: To power the Microprocessor, L.E.D., and Camera
bandage: as a holding surface for the L.E.D. and digital camera.

The Microprocessor can be separately located.

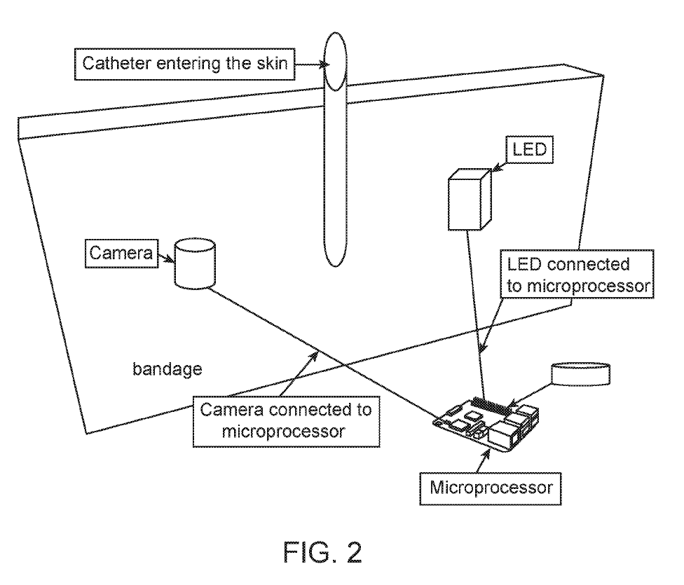

FIG. 2 shows one embodiment of the arrangement, used to measure the bacteria infested neck of the catheter at the patients entry point.

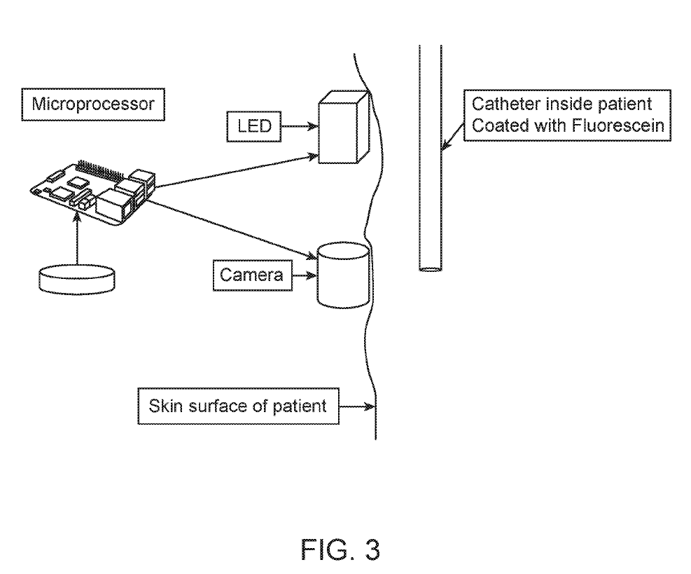

FIG. 3 shows another embodiment, used to measure the bacteria infested catheter internal within the patient.

Without being bound by theory, the microprocessor can periodically turn the L.E.D. on causing it to emit the exact color frequency to excite the exposed flourescein coated catheter which will fluoresce at 513 nano meters. The Digital Camera will record both the image/shape of the catheter and the RGB color pattern emitted by the catheter if it has been deesterized. Using image sensing software that compares this information with image/shape information in its memory bank the microprocessor will be able to locate the exact pixels of the camera corresponding to the catheter. The Microprocessor then can compare the RGB pattern emitted by the catheter to the recorded RGB pattern in its memory bank for Flourescein.

| | R | G | B | nanometer |
|---|---|---|---|---|
| L.E.D. Laser | 0 | 255 | 224 | 493 (blue/green |
| Flourescein flouresces | 21 | 255 | 0 | 513 (green) |

If the camera senses an RGB pattern that matches flourescein within sufficient tolerances, the microprocessor will send a signal, via its Blue Tooth generator, to an external device such as a smartphone, lap top computer, desk top computer, or specialized terminal.

Using a combination of shape detection software and RGB color detection software, the sensitivity of detecting the fluorescence is highly increased, light emitted by spurious sources are depressed, and the interference of the L.E.D. is minimized.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology.

The present disclosure is not to be limited in terms of the particular embodiments described in this application. Many modifications and variations can be made without departing from its spirit and scope, as will be apparent to those skilled in the art.

Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that this disclosure is not limited to particular methods, reagents, compounds, compositions or biological systems, which can of course vary. It is also to be understood that the terminology used herein is for the purpose of describing particular embodiments only, and is not intended to be limiting.

As will be understood by one skilled in the art, for any and all purposes, particularly in terms of providing a written description, all ranges disclosed herein also encompass any and all possible subranges and combinations of subranges thereof. For example, ranges describing isomeric ratios disclosed herein encompass any and all possible subranges of ratios thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EXAMPLES

### Example 1—Prior Art Formation of pH Dependent Liposomes—http://link.springer.com/article/10.1007%2Fs11814-008-0066-6#

pH-sensitive liposomes can be prepared by a detergent removal method. Dioleoylphosphatidylethanolamine (DOPE) and cholesteryl hemisuccinate (CHEMS) are combined with calcein for the preparation of the liposomes so that the molar ratios of DOPE to CHEMS are 9/1, 8/2, 6/4 and 5/5. On transmission electron micrographs, hundreds of nm sized-multilamella vesicles are observed. The degrees of fluorescence (indicator) quenching is approximately 70-80%, indicating that closed vesicles are formed. According to the results of the pH-dependent release experiment with the liposome composed of DOPE/CHEMS (5/5), no significant release is observed in the pH region ranging from 6 to 8. At pH of 5, an appreciable amount of calcein is released. The patterns of pH-dependent releases from liposomes composed of DOPE/CHEMS (6/4) and DOPE/CHEMS (8/2) are almost the same as those from liposomes composed of DOPE/CHEMS (5/5). With the liposomes composed of DOPE/CHEMS (9/1), unlike the other liposomes described above, almost 90% release is observed at pH 6.

The above example demonstrates the formation of liposomes carrying indicators which are designed to degrade at preselected pHs.

Stamping of liposomes onto a polymeric matrix can be achieved by employing a small amount of a solvent system which is first placed on the matrix to dissolve a very fine layer of the polymer. Prior to resolidifying, a liposome composition is then stamped into the liquid layer of the polymer and then allowed to dry. Such stamping allows the polymeric matrix to contain multiple sites of liposome deposits while not having to cover the entirety of the polymeric matrix. In such cases, the matrix is preferably covered with from about 1 to 50 stamps per 10 square centimeters of polymeric film matrix. However, it is understood that more than 50 stamps per 10 square centimeters can be adequately used in this invention.

### Example 2—Conversion of the Fluorescein into a Pro-Fluorescein Attachable Functionality

Fluorescein was converted to its 2,10-di[1,1-dimethylethylcarboxyate)]fluorescein by conversion of the 2,10-diol to the corresponding diester by reaction with an excess pivaloyl chloride in the presence of a base, similar to the procedure described in Scheme 2 below.

### Example 3—Fluorescein Derivatives

The following example illustrates synthesis of compounds which are non-fluorescent but which can be modified to be fluorescent by microbial enzymes. The reactions and procedures are generally known in the art.

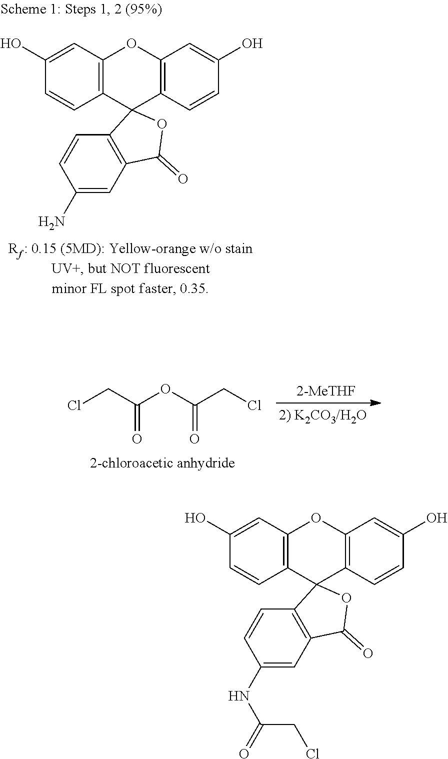 [SMILES: Nc1ccc2c(c1)C(=O)OC21c2ccc(O)cc2Oc2cc(O)ccc21 ; O=C(CCl)OC(=O)CCl ; O=C(CCl)Nc1ccc2c(c1)C(=O)OC21c2ccc(O)cc2Oc2cc(O)ccc21]

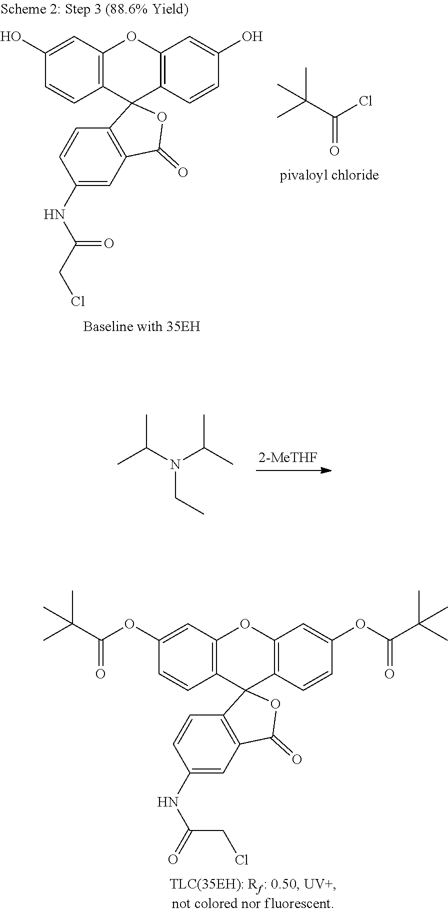

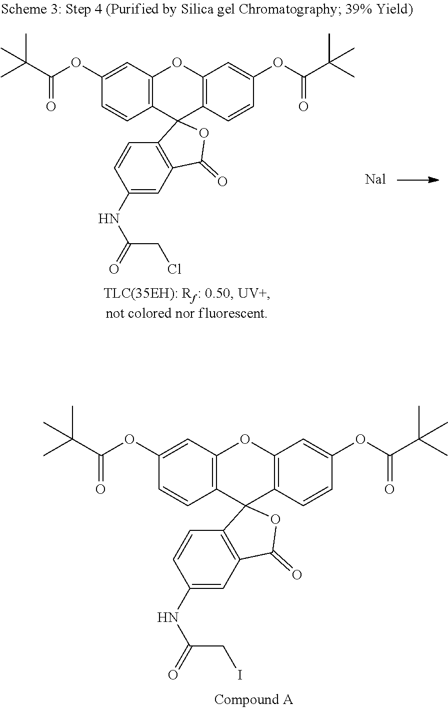

Compound A is but one example of a compound of Formula III or IV which be reacted with hydroxyl or amine functionalities on a polymer, which will replace the iodine and produce an anchored, masked fluorescein moiety. Other compounds of Formula III or IV can also be prepared similarly as described above and used to react with a polymer. Scheme 4 below shows a polymer comprising a fluorescent derivative obtained when the iodine is displaced with an oxygen atom terminating a silicone polymer. An amine group on a resin could also displace the iodine leading to a different anchored compound, shown in Scheme 5. The linker between the fluorescent derivative and the polymer does not hydrolyze because of interactions with the biofilm, but the ester functionality is hydrolyzed in the presence of biofilm enzymes such as esterase, revealing the fluorescent indicator.

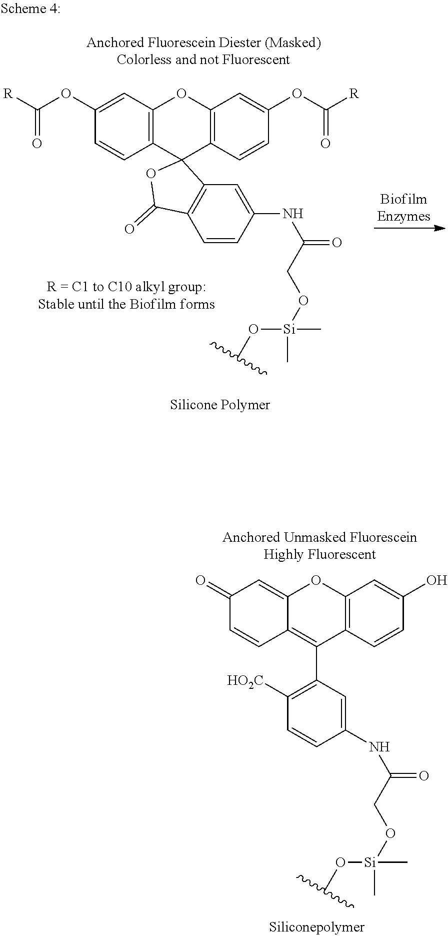

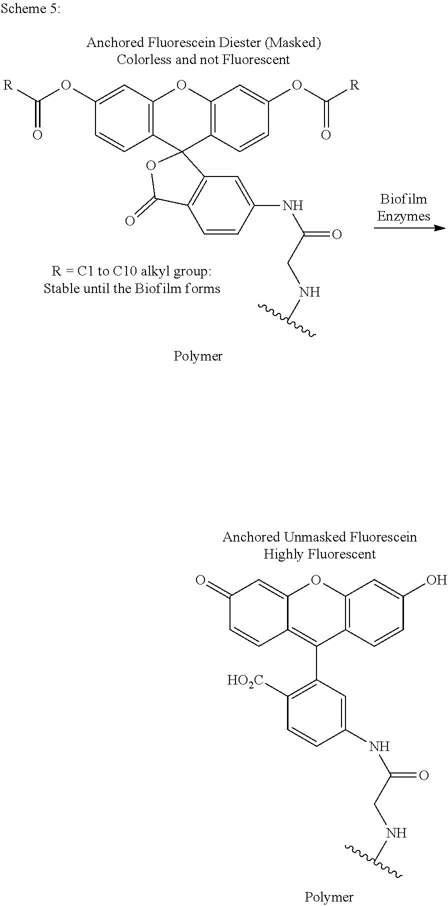

Example 4—Preparation of Fluorescein Derivatives

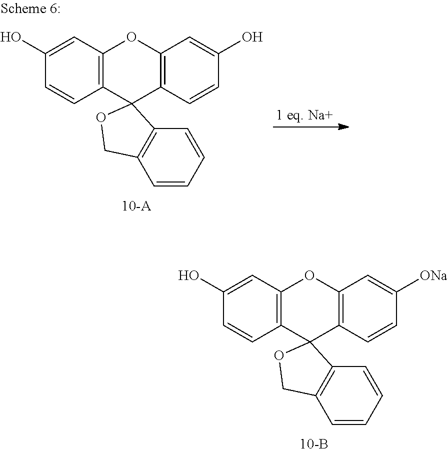

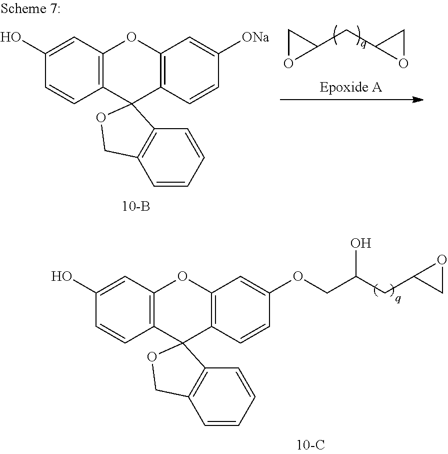

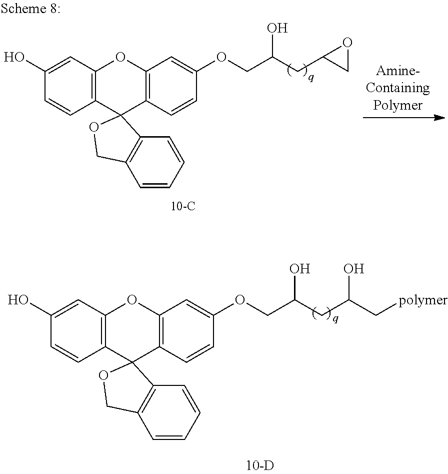

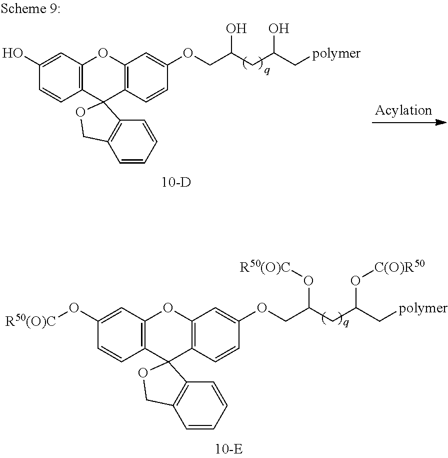

R⁵⁰ is preferably an alkyl or an aryl group.

To prepare ester 10-E, 10-A is reacted with sodium hydroxide to form phenoxide 10-B. Phenoxide 10-B is then reacted with Epoxide A, wherein q is an integer selected, for example, 2, 3, 4, 5, 6, 7, 8, 9, or 10, to form mono-epoxide 10-C. Mono-epoxide 10-C is subsequently reacted with an amine-containing polymer to form polymer 10-D. Polymer 10-D is then acylated to form ester 10-E. Methods of acylation are well-known in the art.

Epoxide A is commercially available or can be readily prepared from an alpha, omega di-epoxide through epoxidation reactions well known in the art.

Other embodiments are set forth in the following claims.

What is claimed is:

1. A compound of Formula V-A or V-A′: 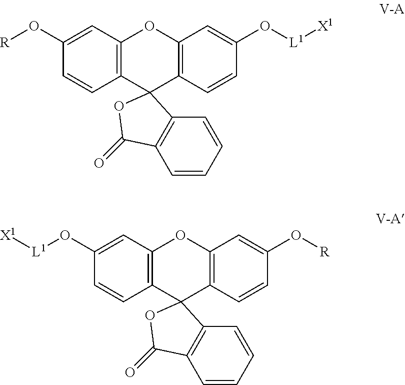
wherein
R is —H, —C(O)(R²⁰), —C(O)O(R²⁰), or —C(O)N(R²¹)₂;
R²⁰ is a C₁-C₃₀ alkyl, C₂-C₃₀ heteroalkyl, C₆-C₁₀ aryl or C₁-C₁₀ heteroaryl;
each R²¹ is independently hydrogen, C₁-C₃₀ alkyl, C₂-C₃₀ heteroalkyl, C₆-C₁₀ aryl or C₁-C₁₀ heteroaryl;
L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate; and
X¹ is a polymer.

2. A compound of Formula V-B: 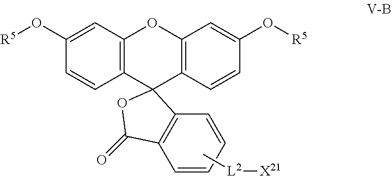
wherein
each R⁵ is independently R or optionally substituted C₁-C₆ alkyl, provided that both R⁵ groups are not optionally substituted C₁-C₆ alkyl;
R is —H, —C(O)(R²⁰), —C(O)O(R²⁰), or —C(O)N(R²¹)₂;
R²⁰ is a C₁-C₃₀ alkyl, C₂-C₃₀ heteroalkyl, C₆-C₁₀ aryl or C₁-C₁₀ heteroaryl;
each R²¹ is independently hydrogen, C₁-C₃₀ alkyl, C₂-C₃₀ heteroalkyl, C₆-C₁₀ aryl or C₁-C₁₀ heteroaryl;
L² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate or L² is a linking group, wherein the linking group is from 1 to 20 carbon atoms and optionally from 1 to 6 heteroatoms selected from the group consisting of oxygen, sulfur, nitrogen, phosphorus and silicon; and
X²¹ is a polymer.

3. A compound of claim 2 of Formula V-B′: 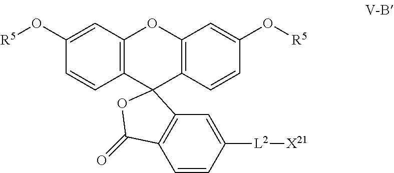
wherein the variables are defined as in claim 2.

4. A compound of Formula V: 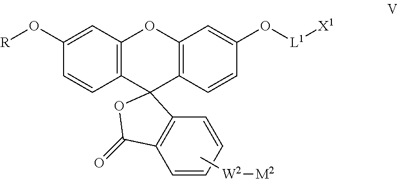
wherein
R is —H, —C(O)(R²⁰), —C(O)O(R²⁰), or —C(O)N(R²¹)₂;
R²⁰ is a C₁-C₃₀ alkyl, C₂-C₃₀ heteroalkyl, C₆-C₁₀ aryl or C₁-C₁₀ heteroaryl;
each R²¹ is independently hydrogen, C₁-C₃₀ alkyl, C₂-C₃₀ heteroalkyl, C₆-C₁₀ aryl or C₁-C₁₀ heteroaryl;
L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate;
X¹ is a polymer;
W² is a linking group, wherein the linking group is from 1 to 20 carbon atoms and optionally from 1 to 6 heteroatoms selected from the group consisting of oxygen, sulfur, nitrogen, phosphorus and silicon or W² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate;
M² is selected from the group consisting of hydrogen, —COOCR¹⁰₃, —COCR¹⁰₃, —C(R¹¹)₃, —C(R¹²)₂—O—R¹³, —COR¹⁴, —Si(R¹⁵)₃, C₁-C₁₀ alkyl group, and a polymer;
each R¹⁰ is independently a C₁-C₆ alkyl optionally substituted with 1, 2 or 3 C₁-C₆ alkoxy groups;
each R¹¹ is independently hydrogen or a phenyl optionally substituted with 1, 2 or 3 substituents independently selected from C₁-C₆ alkyl and C₁-C₆ alkoxy groups;
each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms;
each R¹⁴ is independently a C₁-C₁₀ alkyl; and
each R¹⁵ is independently a C₁-C₃ alkyl.

5. The compound of claim 4, wherein M² is a polymer.

6. The compound of claim 4, wherein M² is a non-polymer substituent.

7. A compound of Formula (I) or (II): 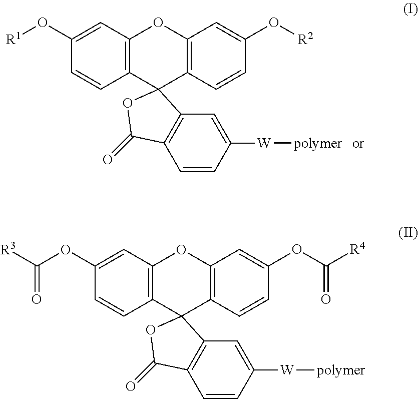
wherein
polymer represents the rest of the polymer molecule,
W is a linking group of from 1 to 20 carbon atoms and optionally from 1 to 6 heteroatoms selected from the group consisting of oxygen, sulfur, nitrogen, phosphorus and silicon,
R¹ and R² are independently H, —COOCR¹⁰₃, —COCR¹⁰₃, —C(R¹¹)₃, —C(R¹²)₂—O—R¹³, —COR¹⁴, or —Si(R¹⁵)₃,
R³ and R⁴ are independently C₁ to C₁₀ alkyl or C₁ to C₁₀ alkoxy,
each R¹⁰ is independently a C₁-C₆ alkyl optionally substituted with 1, 2 or 3 C₁-C₆ alkoxy groups,
each R¹¹ is independently a hydrogen or a phenyl optionally substituted with 1, 2 or 3 substituents independently selected from C₁-C₆ alkyl and C₁-C₆ alkoxy groups, such as methyl and methoxy,
each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms,
each R¹⁴ is independently C₁ to C₁₀ alkyl, and
each R¹⁵ is independently a C₁-C₃ alkyl, such as methyl.

8. A compound of Formula VI-A or VI-A′: 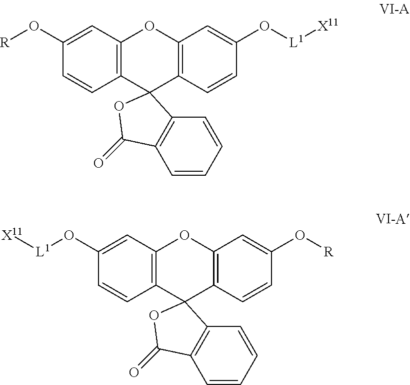
wherein
R is —H, —C(O)(R²⁰), —C(O)O(R²⁰), or —C(O)N(R²¹)₂;
R²⁰ is a C₁-C₃₀ alkyl, C₂-C₃₀ heteroalkyl, C₆-C₁₀ aryl or C₁-C₁₀ heteroaryl;
each R²¹ is independently hydrogen, C₁-C₃₀ alkyl, C₂-C₃₀ heteroalkyl, C₆-C₁₀ aryl or C₁-C₁₀ heteroaryl;
L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate; and
X¹¹ is a reactive functionality that reacts with a complementary group on a polymer.

9. A compound of Formula VI-B: 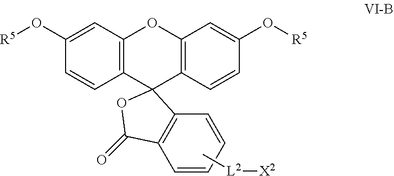
wherein
each R⁵ is independently R or optionally substituted C₁-C₆ alkyl, provided that both R⁵ groups are not optionally substituted C₁-C₆ alkyl;
R is —H, —C(O)(R²⁰), —C(O)O(R²⁰), or —C(O)N(R²¹)₂;
R²⁰ is a C₁-C₃₀ alkyl, C₂-C₃₀ heteroalkyl, C₆-C₁₀ aryl or C₁-C₁₀ heteroaryl;
each R²¹ is independently hydrogen, C₁-C₃₀ alkyl, C₂-C₃₀ heteroalkyl, C₆-C₁₀ aryl or C₁-C₁₀ heteroaryl;
L² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate or L² is a linking group, wherein the linking group is from 1 to 20 carbon atoms and optionally from 1 to 6 heteroatoms selected from the group consisting of oxygen, sulfur, nitrogen, phosphorus and silicon; and
X² is a reactive functionality that reacts with a complementary group on a polymer.

10. A compound of Formula VI: 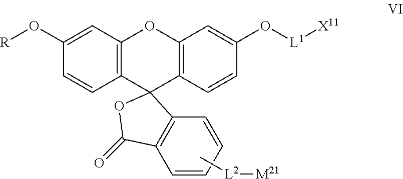
wherein
R is —H, —C(O)(R²⁰), —C(O)O(R²⁰), or —C(O)N(R²¹)₂;
R²⁰ is a C₁-C₃₀ alkyl, C₂-C₃₀ heteroalkyl, C₆-C₁₀ aryl or C₁-C₁₀ heteroaryl;
each R²¹ is independently hydrogen, C₁-C₃₀ alkyl, C₂-C₃₀ heteroalkyl, C₆-C₁₀ aryl or C₁-C₁₀ heteroaryl;
L¹ is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate;
X¹¹ is a reactive functionality that reacts with a complementary group on a polymer;
L² is a C₁-C₃₀ alkylene or C₂-C₃₀ heteroalkylene optionally substituted with 1, 2 or 3 hydroxy groups, wherein the hydroxy groups can be further derivatized to an alkoxy, a carboxylate ester, a keto, or a carbamate or L² is a linking group, wherein the linking group is from 1 to 20 carbon atoms and optionally from 1 to 6 heteroatoms selected from the group consisting of oxygen, sulfur, nitrogen, phosphorus and silicon;
M²¹ is selected from the group consisting of hydrogen, —COOCR¹⁰₃, —COCR¹⁰₃, —C(R¹¹)₃, —C(R¹²)₂—O—R¹³, —COR¹⁴, —Si(R¹⁵)₃, C₁-C₁₀ alkyl group, and a reactive functionality that reacts with a complementary group on a polymer;
each R¹¹ is independently a hydrogen or a phenyl optionally substituted with 1, 2 or 3 substituents independently selected from C₁-C₆ alkyl and C₁-C₆ alkoxy groups;
each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms;
each R¹⁴ is independently a C₁-C₁₀ alkyl; and
each R¹⁵ is independently a C₁-C₃ alkyl.

11. The compound of claim 10, wherein M²¹ is a reactive functionality that reacts with a complementary group on a polymer.

12. The compound of claim 10, wherein M²¹ is a group other than a reactive functionality that reacts with a complementary group on a polymer.

13. The compound of any one of claims 1-6 and 8-12, wherein R is —H.

14. The compound of any one of claims 1-6 and 8-12, wherein R is —C(O)(R²⁰).

15. The compound of any one of claims 1-6 and 8-12, wherein R is —C(O)O(R²⁰).

16. The compound of any one of claims 1-6 and 8-12, wherein R is —C(O)N(R²¹)₂.

17. The compound of any one of claims 1-6 and 8-12, wherein R²⁰ is a C₄-C₃₀ alkyl.

18. The compound of any one of claims 1-6 and 8-12, wherein R²⁰ is a C₈-C₃₀ alkyl.

19. The compound of any one of claims 1-6 and 8-12, wherein R²⁰ is a C₁₀-C₃₀ alkyl.

20. The compound of any one of claims 1-6 and 8-12, wherein R²⁰ is a C₁₂-C₃₀ alkyl.

21. A compound of Formula (III) or (IV): 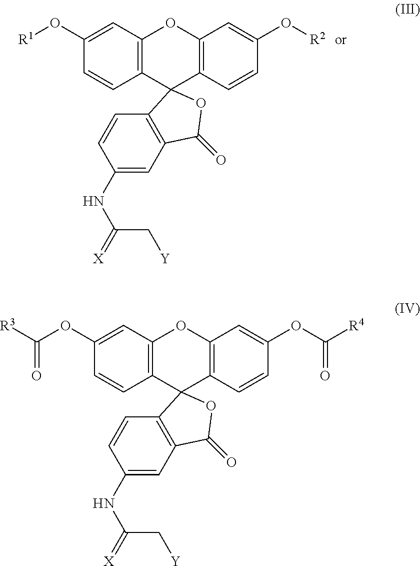
where
X is sulfur or oxygen,
Y is chloro, bromo or iodo,
R¹ and R² are independently H, —COOCR¹⁰₃, —COCR¹⁰₃, —C(R¹¹)₃, —C(R¹²)₂—O—R¹³, —COR¹⁴, or —Si(R¹⁵)₃,
R³ and R⁴ are independently C₁ to C₁₀ alkyl or C₁ to C₁₀ alkoxy,
each R¹⁰ is independently a C₁-C₆ alkyl optionally substituted with 1, 2 or 3 C₁-C₆ alkoxy groups,
each R¹¹ is independently a hydrogen or a phenyl optionally substituted with 1, 2 or 3 substituents independently selected from C₁-C₆ alkyl and C₁-C₆ alkoxy groups, such as methyl and methoxy,
each R¹² is independently hydrogen or a C₁-C₆ alkyl and each R¹³ is independently a hydrogen, a C₁-C₆ alkyl or phenyl optionally substituted with C₁-C₆ alkyl, C₁-C₆ alkoxy or hydroxyl, or R¹² and R¹³ together with the atoms to which they are attached form a 5 or 6 membered heterocyclic ring comprising carbon ring atoms and 1 or 2 oxygen ring atoms,
each R¹⁴ is independently C₁ to C₁₀ alkyl, and
each R¹⁵ is independently a C₁-C₃ alkyl, such as methyl.

22. The compound of claim 7 or 21, wherein R¹ and R² are independently —C(R¹¹)₃, and each R¹¹ is independently a phenyl optionally substituted with one methyl group.

23. The compound of claim 7 or 21, wherein R¹ and R² are independently selected from —CO—O—C(CH₃)₃, —OCH₂—O—CH₃, (p-methoxyphenyl)diphenylmethyl ether 4′-methoxytrityl (MMTr), di-(p-methoxyphenyl)phenylmethyl ether (4′,4′-dimethoxytrityl or DMTr), tri-(p-methoxyphenyl)methyl ether (4′,4′,4′-trimethoxytrityl or TMTr) and

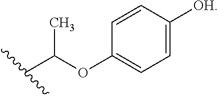 [SMILES: *C(C)Oc1ccc(O)cc1]

24. The compound of claim 7 or 21, wherein R³ and R⁴ are independently methyl, methoxy, t-butyl or t-butoxy.

25. The compound of claim 7 or 21, wherein R¹ and R² are non-hydrogen substituents.
